(19) United States
(12) Patent Application Publication
(10) Pub. No.: US 2012/0289935 A1
(43) Pub. Date: Nov. 15, 2012

(54) TOOL FOR MANIPULATING AN OBJECT IN A BODY CAVITY

(76) Inventors: Hans Almer Middelbeek, Boxmeer (NL); Jozefus A.C. Smit, Boxmeer (NL); Marc-Antoine Driancourt, Beaucouze Cedex (FR); Angella Prohaczik, Beaucouze Cedex (FR)

(21) Appl. No.: 13/522,371

(22) PCT Filed: Jan. 17, 2011
(86) PCT No.: PCT/EP11/50499

§ 371 Date: Jul. 16, 2012

Related U.S. Application Data
(60) Provisional application No. 61/296,581, filed on Jan. 20, 2010.

(30) Foreign Application Priority Data
Jan. 18, 2010 (EP) 10150929.7

(51) Int. Cl. A61M 31/00; A61B 17/00

(52) U.S. Cl. 604/514; 606/1; 604/285

(57) ABSTRACT

A tool for manipulating a solid object (12) in a cavity (16) of a human or animal body, comprising a longitudinal rod member (14) adapted for insertion into said body cavity (16), a first coupling member (26) connected to one of the rod member (14) and the object (12) through a flexible filament (28), and a second coupling member (24) connected to the other of the rod member and the object, said first and second coupling members being arranged for being mechanically coupled together through magnetic attraction, wherein one of the first and second coupling members forms a recess (30), the other of the first and second coupling members is configured as a key member (26), and magnetic poles of at least one of the first and second coupling members are arranged for drawing the key member into the recess along a direction perpendicular to the longitudinal direction of the rod member, wherein the key member and recess are constituted such that when the key member is drawn into the recess, an operative contact surface exists between the key member and the recess, the said contact surface extending in a direction perpendicular to the longitudinal direction of the rod member.

The invention relates to a tool for manipulating a solid object in a cavity of a human or animal body, comprising a rod member adapted for insertion into said body cavity, a first coupling member connected to one of the rod member and the object through a flexible filament, and a second coupling member connected to the other of the rod member and the object, said first and second coupling members being arranged for being mechanically coupled together through magnetic attraction.

The invention also relates to a drug administration set comprising such a tool.

Further, the invention relates to a method of drug administration.

More particularly, the invention is concerned with drug administration to non-human mammals, wherein the object to be manipulated is an intravaginal device, for example for administering drugs for controlling the reproductive cycle of a sow or gilt.

WO 2008/061963 A2 discloses a helically shaped drug supporting anchor that comprises a layered polymer fibre. The drug is contained in one of the layers of the fibre. The fibre is wound to a helix the outer diameter of which is slightly larger than the internal diameter of the vaginal cavity of the mammal to be treated, so that, thanks to its own restoration force, the helix will tend to expand and firmly engage the internal walls of the vaginal cavity so as to be retained therein. When the helix is retained in the vaginal cavity in this way, the drug will gradually be released from the fibre in suitable amounts.

U.S. Pat. No. 3,892,238 discloses a drug administration tool comprising a helically shaped drug supporting anchor and an insertion rod around which a fibre or string forming the drug supporting anchor is wound helically. The insertion rod has means for applying an additional torsional strain to the helix, so that the curvature of the fibre is increased and the outer diameter of the helix is reduced when the same is introduced in the vaginal cavity. Then, when the helix is in place, the torsional strain is relieved, so that the helix resumes its greater original diameter and is firmly held in the vaginal cavity.

After treatment, the drug supporting anchor has to be withdrawn from the vaginal cavity. It is essential that the withdrawal process can be performed with high reliability, because a too long drug exposure and/or a permanent presence of the drug supporting anchor in the vagina would be disadvantageous, e.g. because it would not allow breeding of the animal.

U.S. Pat. No. 3,805,777 discloses an intrauterine device in the form of an annular object, as well as a tool for withdrawing the annular object from the uterus. This tool uses magnetic attraction of the two coupling members for withdrawing the object.

U.S. Pat. No. 6,551,304 discloses a longitudinal apparatus for retrieving a remotely located device, in particular present in a cavity of the human body, using magnetic coupling forces. The magnetic poles are arranged such that a key member connected to the device is drawn into a recess of the apparatus along a longitudinal direction of the apparatus. The longitudinal direction of the apparatus coincides with the retrieval—and hence pulling—direction of the device. Given the fact that magnetic forces are too weak to ensure that the device remains coupled to the apparatus during retrieval, elements are provided at the distal end of the longitudinal apparatus to ensure mechanical grasping and locking of the key member. Comparable apparatus, employing elements to grasp and lock the key member are also known from U.S. Pat. No. 3,786,807 and EP 276 104.

DE 3403294 describes a tool which has the configuration described in the preamble of claim 1. The tool comprises a rod which has a plurality of shields to mechanically lock spherical key members when retrieving an intra-uterine device. The magnetic poles of the tool are arranged for drawing the spherical key member into the recess along a direction perpendicular to the longitudinal direction of the rod member. In order to ensure that the spherical key members remain locked behind the shields when retrieving the device, the shields are constituted as rigid curved shields. These shields therefore constitute barbs that may easily damage the internal wall of the uterus and cervix.

It is an object of the invention to provide a tool that permits to manipulate (e.g. to insert or extract) the object in the body cavity with high reliability and with a low risk of damaging the internal walls of the cavity.

In order to achieve this object, the tool according to the invention is characterised in that the key member and recess are constituted such that when the key member is drawn into the recess, an operative contact surface exists between the key member and the recess, the said contact surface extending in a direction perpendicular to the longitudinal direction of the rod member.

When the key member is accommodated in the recess, and the rod member is moved in a direction normal to the direction in which the recess opens, the two coupling members will be held together not only by magnetic attraction but also by form fit of the key in the recess, so that the forces transmitted from the rod member to the object can be significantly larger than the magnetic forces between the coupling members. It was surprisingly found that the use of an operative contact surface which extends in a direction perpendicular to the longitudinal direction of the rod member, instead of a contact point as known from the prior art (in particular DE 3403294), takes the need away to constitute the recess in the form of a curved shield. This significantly reduces the risk of damaging the internal walls of the body cavity. When the mechanical coupling is to be established, it is sufficient to bring the rod member into the vicinity of the object, so that the coupling members will magnetically attract one another. The flexible filament will permit the first coupling member to move under the influence of the magnetic forces and to automatically assume a posture in which the key member fits into the recess.

More specific optional features of the invention are indicated in the dependent claims.

The first coupling member may be connected, via the flexible filament, to the object whereas the second coupling member is formed at a distal end of the rod member, or vice versa. Similarly, the recess may be formed in the first coupling member while the second coupling member forms the key member, or vice versa.

When the recess is formed in the rod member, it is preferable that the recess opens in a lateral direction of the rod member, so that form fit between the recess and the key member will occur when the rod is moved in its longitudinal direction.

When the key member is the first coupling member, it is preferable that the second coupling member has a slot that opens into the recess and permits the flexible filament to pass through in a direction essentially in parallel with the plane of the opening of the recess.

In one embodiment, both, the first and the second coupling members are permanent magnets. In another embodiment, one of the permanent magnets, preferably the one connected to the rod member, may be replaced by an electro-magnet, so that magnetic attraction can be switched on and off. A coil of the electro-magnet may also be used for detecting whether the key member is properly accommodated in the recess, because the presence of the key member will cause a significant change in the inductivity of the coil. As an alternative, a separate sensor coil may be provided for that purpose.

In yet another embodiment, one of the first and second coupling members may be a permanent magnet or an electro-magnet, whereas the other coupling member is just a piece of magnetisable material.

A drug administration set according to the invention comprises the tool that has been described above, and a drug supporting anchor as the object to be manipulated. The drug supporting anchor may have a helical shape adapted to insertion into the vagina of an animal, and may be made of a material having a shape memory property. Then, the set will preferably comprise an insertion tool for inserting the drug supporting anchor into the vagina, said insertion tool being essentially formed by a rigid tube which accommodates the drug supporting anchor and confines the same to an extended configuration with reduced curvature. As a result, the tool may have a small outer diameter so as to avoid an injury of the animal during the insertion process. When the tube has been inserted into the vagina, the rod member of the withdrawal tool may be used for pushing-out the drug supporting anchor out through the distal end of the tube, whereupon the drug supporting anchor, thanks to its shape memory property, will re-assume its original helical shape, with the windings of the helix closely engaging the walls of the vagina.

In another embodiment, especially when the magnet in one of the coupling members is an electro-magnet that can be switched on and off, the rod member may also be used for inserting the helical drug supporting anchor into the vagina. For example, the helical drug supporting anchor may be provided with two magnetic key members connected to the opposite ends of the helix via respective flexible filaments. Then, the key member at the distal end of the helix may be used for inserting the drug supporting anchor into the vagina. When the electro-magnet is switched off, the rod member may be withdrawn whereas the drug supporting anchor remains in the vagina. Then, when the drug supporting anchor is to be removed again, the rod member will be inserted once again and the magnet will be switched on when the tip end of the rod member reaches the vicinity of the proximal end of the helix, so that the key member attached to that end will engage in the recess of the rod member.

Preferred embodiments will now be explained in conjunction with the drawings, wherein:

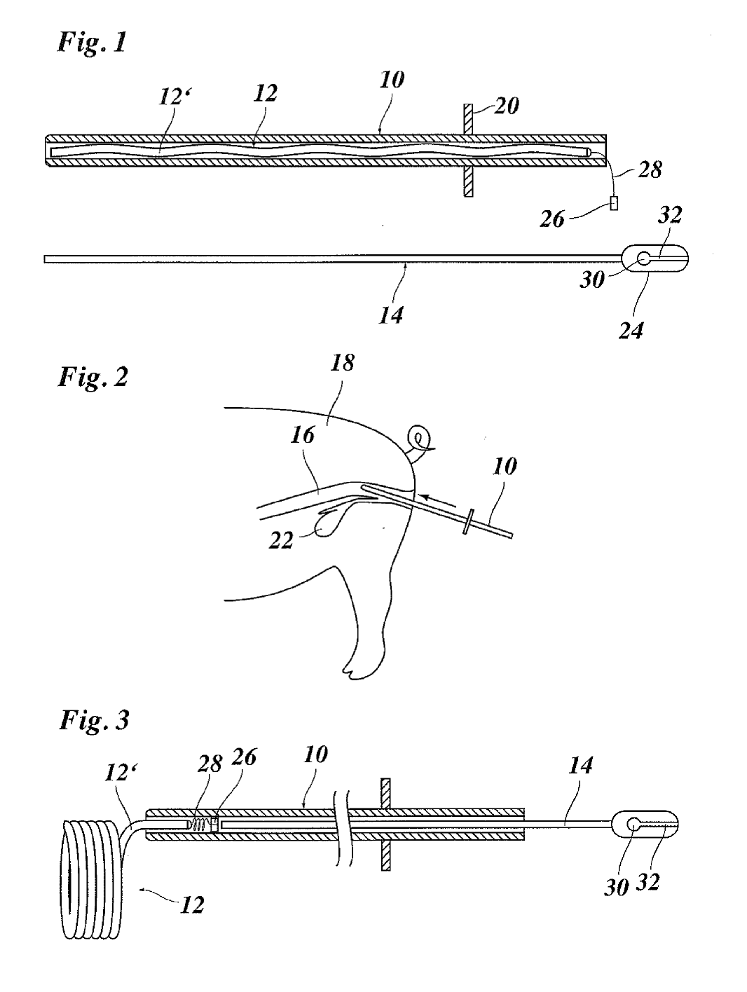

FIG. 1 is a longitudinal sectional view of a drug administration set in a state ready for drug administration;

FIG. 2 is a sketch of the vaginal track of a sow or gilt, illustrating a drug administration method;

FIG. 3 shows the drug administration set in a state in an end phase of a process of inserting a drug supporting anchor into the vagina;

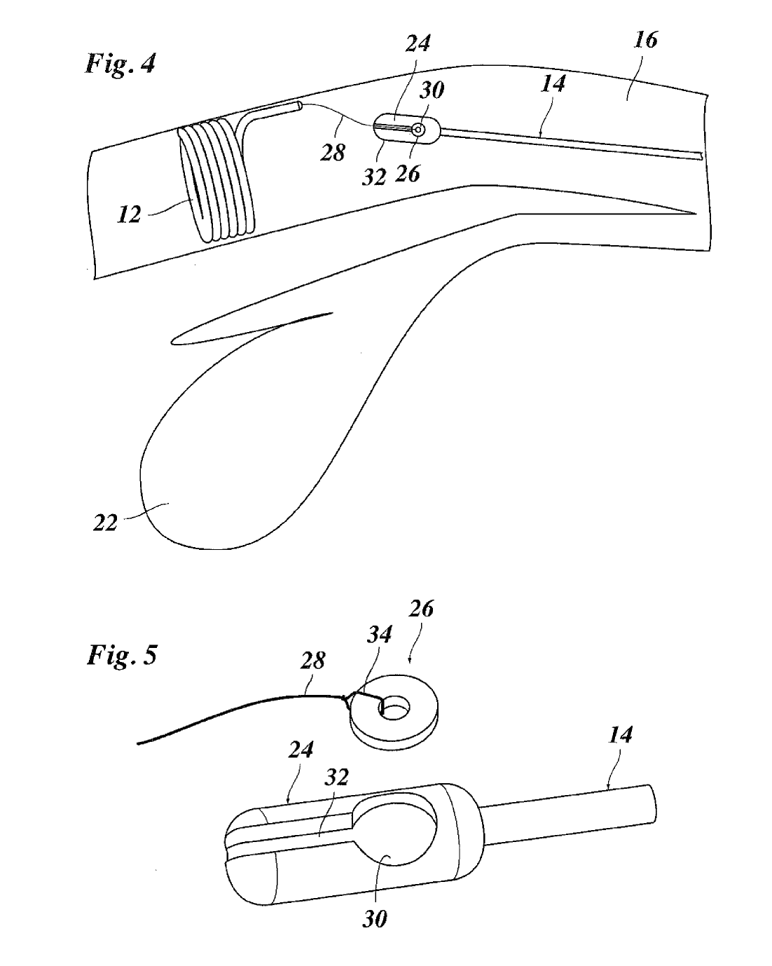

FIG. 4 is an enlarged sketch of the vaginal track and shows a tool according to the invention for removing the drug supporting anchor;

FIG. 5 is an enlarged perspective view of the tool shown in FIG. 4;

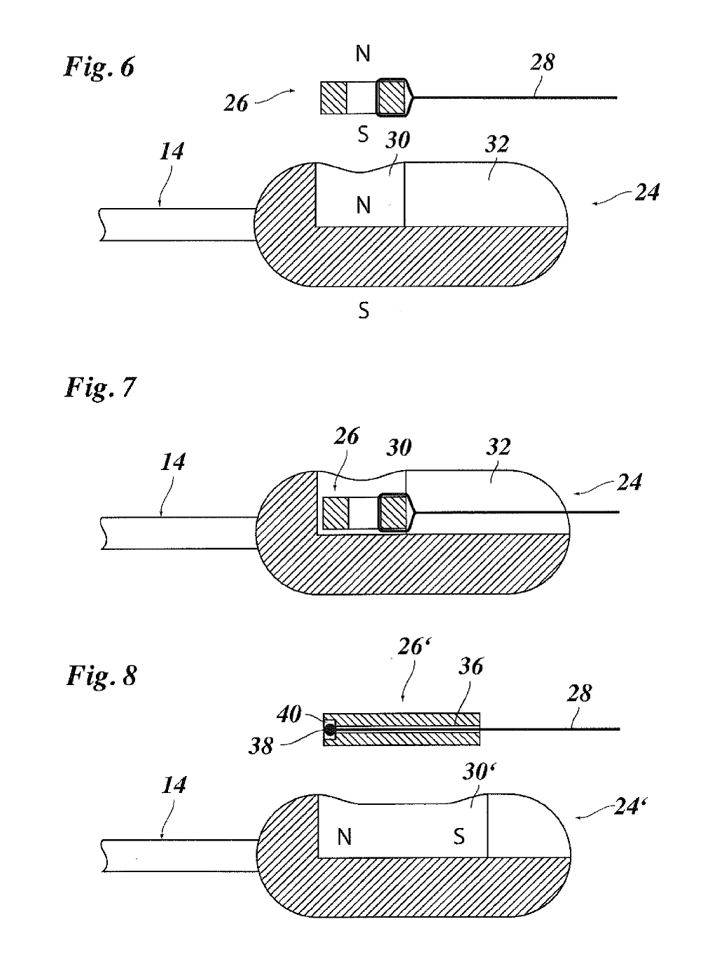

FIGS. 6 and 7 show longitudinal sections of the tool shown in FIGS. 4 and 5 in different conditions;

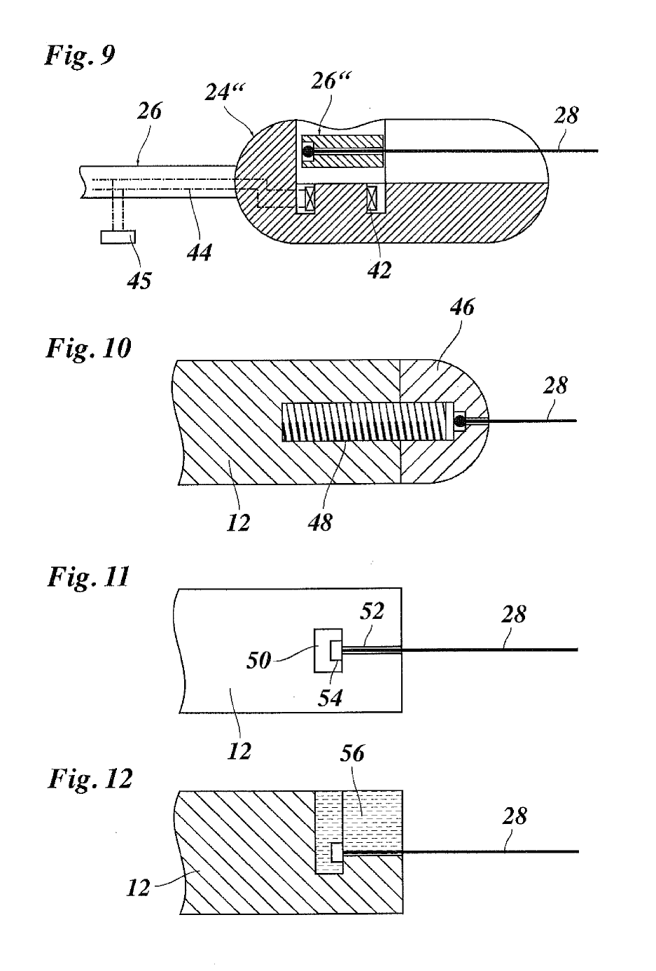

FIGS. 8 and 9 show longitudinal sections of modified embodiments of the tool; and

FIGS. 10 to 13 illustrate different structures for attaching a coupling member to one end of the drug supporting anchor.

FIG. 14 illustrates the operative contact surface of the present invention versus the contact point of a prior art device.

As is shown in FIG. 1 a drug administration set according to the invention comprises three main components: a rigid tube 10, a drug supporting anchor 12 accommodated in the tube 10, and a rod member 14.

The tube 10 may be made of metal or plastics and is rounded at its tip end (on the left side in FIG. 1) and is generally configured to be inserted into a body cavity, e.g. the vagina 16 of a gilt 18, as is shown in FIG. 2. The tube has a disk-shaped handle 20 which facilitates the processes of insertion into and extraction from the vagina 16. As is shown in FIG. 2, the tube 10 is preferably inserted in a slightly upwardly inclined position in order to prevent the tube from entering into the bladder 22 rather than the vagina 16 of the gilt.

The drug supporting anchor 12 is formed by a fibre or an extruded semi-rigid string 12′ of a polymer material that is impregnated with the drug to be administered to the gilt 18, e.g. a drug for controlling the reproductive cycle of the animal. The natural shape of the drug supporting anchor 12, i.e. the shape it assumes when it is not subject to any external forces, is that of a helical spring (a helix) the outer diameter of which is slightly larger than the internal diameter of the vagina 16 of the gilt. In the condition shown in FIG. 1, however, the internal walls of the tube 10 prevent the drug supporting anchor 12 from assuming its natural shape, and the string 12′ is confined to assume an extended shape, in which its natural curvature is reduced to minor warps. Preferably, the internal diameter of the tube 10 is only slightly larger than the external diameter of the string forming the drug supporting anchor 12. For example, the internal diameter of the tube 10 is less than twice the external diameter of the string 12′, as in FIG. 1.

When the tube 10 has been inserted into the vagina 16 of the gilt 18, with the drug supporting anchor 12 accommodated in the tube, the rod member 14 will be inserted into the tube 10 from the rear end, so that the drug supporting anchor 12 is pushed out from the tip end of the tube. In the example shown, the rod member 14 has a cigar-shaped handle 24 which facilitates the manipulation of the rod member but has also another function that will be explained later.

FIG. 3 shows a condition where the drug supporting anchor 12 has almost completely been pushed out of the tube 10. Since the material of the string 12′ forming the drug supporting anchor 12 is elastic or has at least a certain shape memory property, it resumes its helical natural shape as soon as it has left the tube 10. As a consequence, the helical drug supporting anchor 12 will seat itself in the vagina 16, with its outer periphery engaging the internal walls of the vagina with light pressure, so that the drug supporting anchor will safely be retained in position when the tube 10 and the rod member 14 are withdrawn.

The rod member 14 is not only used for pushing the drug supporting anchor 12 out of the tube 10, but forms also part of a tool for withdrawing the drug supporting anchor 12 from the vagina 16. As is shown in FIGS. 1 and 3, a key member 26 (first coupling member) is connected to the proximal end of the drug supporting anchor 12 through a flexible filament 28, e.g. a thread with high tensile strength. The handle 24 at one end of the rod member 14 is configured as a second coupling member and forms a recess 30 with a shape suitable for the key member 26 to fit in. A slot 32 extends longitudinally through the handle 34 and connects the recess 30 to the tip end of the handle.

As is shown in FIG. 3, the key member 26 has a diameter smaller than the internal diameter of the tube 10, so that the key member 26 and the filament 28 that are fixedly connected to the drug supporting anchor 12 may be pushed through the tube 10 together with the drug supporting anchor by means of the rod member 14 serving as a pushed-rod. Please note that the filament, together with key member 26, may also be connected to the proximal end of anchor 12. This has the advantage that key member 26 does not need to fit into tube 10.

FIG. 4 illustrates how the drug supporting anchor 12 is withdrawn from the vagina by means of the rod member 14. To that end, the rod member 14 is inserted into the vagina 16 in reverse orientation, i.e. with the handle 24 forming the distal end. The handle 24 and the key member 26 include permanent magnets (or alternatively, if appropriate may be formed by such magnets), so that they magnetically attract one another. In the example shown, the key member 26 has an annular shape that smoothly fits into the recess 30 of the handle 24. The handle and the key member are magnetised such that a pole formed at the bottom of the recess 30 attracts one end face of the key member 26. Thus, as soon as the handle 24 reaches the vicinity of the proximal end of the drug supporting anchor 12, the key member 26 will be attracted towards the handle 24. The flexible filament 28 permits the necessary movement of the key member 26 and also permits the key member to assume an orientation in which it is drawn into the cavity 30 by the magnetic forces.

When, now, the rod member 14 is withdrawn, the form fit between the key member 36 and the recess 30 assures that the force exerted onto the rod member 14 is transmitted to the key member 26 via the peripheral wall of the recess 30. As a result, the filament 28 is subject to a tensile force which causes the same to settle in the slot 32 of the handle 24 and to transmit the tensile force to the proximal end of the drug supporting anchor 12. As a result, the helical drug supporting anchor 12 will be drawn out to assume a stretched configuration with smaller diameter, so that it may smoothly be drawn out of the vagina 16.

FIG. 5 shows the handle 24 at the end of the rod member 14 and the key member 26 in a perspective view. The flexible filament 28 may safely be secured to the key member 26 by forming a loop 34 that passes through the central hole of the annular key member 26.

The recess 30 in the handle 24 has a flat circular bottom face against which the key member 26 will be attracted. The circular contours of the recess 30 and the key member 26 permit the key member to rotate until the filament 28 is accommodated in the slot 32. Then, the peripheral wall of the recess 30 may exert a force onto the key member 26 tending to tension the filament 28, and the reaction force of the filament 28 will draw the key member 26 closer against the peripheral wall of the recess 30 in the direction in parallel with the longitudinal direction of the rod member 14 and the slot 32 and normal to the peripheral wall of the recess 30, so that the filament will not cause the key member 26 to be drawn out of the recess. In a preferred embodiment two recesses 30 are present in handle 24, one recess on either side of the magnet present in the hart of handle 24.

FIG. 6 shows the handle 24 of the rod member 14 as well as the key member 26 in respective longitudinal sectional views in a condition in which the key member is about to be drawn into the recess 30. In this example, the handle 24 is magnetised such that the bottom surface of the recess 30 forms a magnetic north pole N and the peripheral surface of the handle opposite to the recess 30 forms a magnetic south pole S, and the annular key member 26 is magnetised such that its north and south poles N, S are formed by the opposite end faces. As a result, magnetic forces will automatically cause the key member 26 to rotate into an orientation in which its south pole S faces the north pole N at the bottom of the recess 30, and will then be drawn into the recess, as shown in FIG. 7.

FIG. 8 illustrates a modified embodiment in which the first coupling member is formed by a key member 26′ that is not a permanent magnet but just a piece of magnetisable material, e.g. iron. It is preferable that the magnetic or magnetisable material of the key member 26, 26′ is coated by a suitable coating, e.g. a plastic coating that can easily be cleaned and desinfected.

In the example shown in FIG. 8, the key member 26′ has an elongated rod-like shape, and the filament 28 passes through an axial bore 36 of the key member and has a thickened end 38 accommodated in a recess 40 at one end of the key member.

The second coupling member is formed by a handle 24′ having an elongated recess 30′ that extends in longitudinal direction of the rod member 14 and is adapted to the elongated shape of the key member 26′. The handle 24′ is a permanent magnet that is magnetised such that its north and south poles N, S are formed by opposite axial ends of the elongated recess 30′.

When the key member 26′ is brought into the vicinity of the handle 24′, the elongated shape of the key member 26′ may cause the same to assume the orientation of the magnetic field around the recess 30′, so that the key member will fit into the recess. Further, the magnetic field will magnetise the material of the key member 26′, so that the latter is attracted into the recess.

In principle, it is possible in the embodiment shown in FIG. 8, that the key member 26′ is drawn into the recess 30′ in the wrong orientation, i.e. with the filament 28 exiting to the opposite side. However, this is unlikely to happen, because when the handle 24′ is brought into the vicinity of the drug supporting anchor 12, the key member 26′ will be attracted towards the rod member 14 that has been inserted into the vagina, and the filament 28 will be stretched and will exert a tensile force onto the key member 26 causing the same to assume the posture shown in FIG. 8.

FIG. 9 shows yet another embodiment in which the first coupling member is formed by a key member 26″ that is shaped as a circular disk (shown in cross-section in FIG. 9), and the second coupling member is formed by a handle 24″ similar to the handle 24 shown in FIGS. 1 to 7, with the difference that it does not include a permanent magnet but instead includes an electro-magnet 42 that is powered via electrical leads 44 that pass through the rod member 14. Thus, the magnetic attraction between the coupling members may be switched on and off as desired.

The leads 44 are connected to a current source and switch (both not shown) and are further connected to a detection and indication device 45 that measures the inductivity of the coil forming the electro-magnet 42. If the key member 26″ is not properly seated in the recess of the handle 24″, then the inductivity will be smaller than usual, and this is indicated to the user by an acoustic or optical warning signal, e. g. by lighting an LED.

The key member 26″ is attached to the filament 28 in the same manner as in FIG. 8, but may be a permanent magnet as in FIG. 7.

In yet another embodiment, the key member 26′ shown in FIG. 8 (no permanent magnet) may be combined with a handle similar to the handle 24′ shown in FIG. 8 but having one or two electro-magnets for generating the magnetic field.

FIG. 10 is an enlarged cross-sectional view of the proximal end of the helical drug supporting anchor 12 that is connected to the filament 28. By means of a structure that is similar to the one shown in FIGS. 8 and 9, the filament 28 is attached to a cap 46 that rounds-off the end of the drug supporting anchor 12 and is connected to the latter by a threaded bolt 48. The bolt 48 has a first end embedded in the plastic material of the drug supporting anchor 12 and a second end projecting out of the drug supporting anchor. The cap 46 has an internally threaded bore and is screwed onto the projecting end of the bolt 48.

In a modified embodiment, the bolt 48 may be formed in one piece with the cap 46 or embedded therein, and may have self-cutting threads to be screwed into the drug supporting anchor 12.

In yet another embodiment, the filament 28 may form a loop that passes through a transversal through-bore of the drug supporting anchor 12.

FIGS. 11 and 12 illustrate still another embodiment in which the drug supporting anchor 12 has a recess 50 that is open towards the peripheral surface of the string forming the drug supporting anchor 12. The recess 50 is spaced apart from the proximal end of the drug supporting anchor 12 and is connected to that proximal end by a longitudinal slot 52. The filament 28 has a thickened end 54 accommodated in the recess 50, and an adjacent end portion of the filament passes through the slot 52. When the filament 28 has been hooked-in at the drug supporting anchor 12 in this way, the recess 50 and the slot 52 are filled with an adhesive or resin 56, as shown in FIG. 12, so that the filament and its thickened end are secured in position.

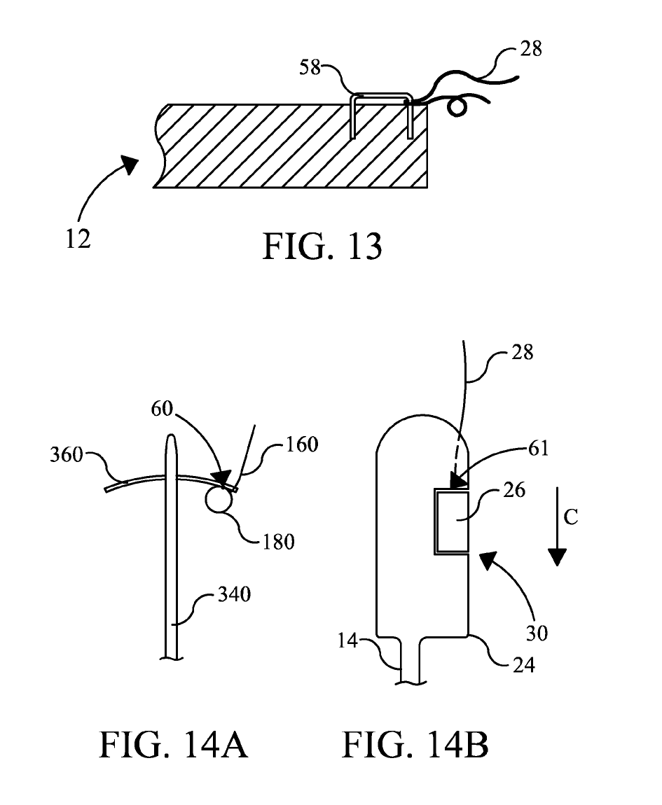

FIG. 13 shows an embodiment wherein the filament 28 forms a loop that passes through a U-shaped clamp 58 that has simply been punched into the end of the drug supporting anchor 12 from the peripheral surface thereof.

Finally, FIG. 14 illustrates the operative contact surface of the present invention versus the contact point of the prior art device as known from DE 3403294. The begin with the latter in FIG. 14A, the key member 180 is spherical and hence, when drawn into the recess formed by curved shield 360 (attached to rod member 340), merely has a contact point 60 with shield 360. Since upon pulling the rod member 340 out of the body cavity there will always be pulling forces in a direction perpendicular to the longitudinal direction of the rod member 340, in particular through the inherent slight lateral position of filament 160, the shield 360 is formed as a rigid curved shield, to ensure a reliable mechanical coupling between the intrauterine device (not shown in FIG. 14A) and rod 340.

In contrast, in the present invention (FIG. 14B) key member 26 and recess 30 share a common operative contact surface 61. This (curved) surface extends in a direction perpendicular to the longitudinal direction of the rod member 14. It was found that this constitution provides a reliable coupling between the key member 26 and recess 30, despite the fact that some pulling force perpendicular to the longitudinal axis of the rid member 14 will inherently be present when pulling the rod member out of a body cavity. Still, the presence of this operative contact surface takes the need away to form the recess in the form of a barb. It is noted that actual contact between the key member 26 and a wall of the recess 30 at operative contact surface 61 will of course be formed not until a pulling force is exerted on the rod member in the indicated direction C. It is also noted that this actual contact on a micrometer or even molecular scale may be restricted to no more then a few contact points.

1. A tool for manipulating a solid object (12) in a cavity (16) of a human or animal body, comprising a longitudinal rod member (14) adapted for insertion into said body cavity (16), a first coupling member (26; 26′; 26″) connected to one of the rod member (14) and the object (12) through a flexible filament (28), and a second coupling member (24; 24′; 24″) connected to the other of the rod member and the object, said first and second coupling members being arranged for being mechanically coupled together through magnetic attraction, wherein one of the first and second coupling members forms a recess (30; 30′), the other of the first and second coupling members is configured as a key member (26; 26′; 26″), and magnetic poles (N, S) of at least one of the first and second coupling members are arranged for drawing the key member into the recess along a direction perpendicular to the longitudinal direction of the rod member, characterised in that the key member and recess are constituted such that when the key member is drawn into the recess, an operative contact surface (61) exists between the key member and the recess, the said contact surface extending in a direction perpendicular to the longitudinal direction of the rod member.

2. The tool according to claim 1, wherein the first coupling member (26; 26′; 26″) is connected to the object (12).

3. The tool according to claim 2, wherein the second coupling member (24; 24′; 24″) forms the recess (30; 30′).

4. The tool according to claims 3, wherein the second coupling member (24; 24′; 24″) is rigidly connected to the rod member (14), and the recess (30; 30′) opens laterally of the rod member (14).

5. The tool according to claim 4, wherein each of the first and second coupling members (24, 26) includes a permanent magnet.

6. The tool according to claim 4, wherein at least one of the first and second coupling members includes an electro-magnet (42).

7. The tool according to claim 5, wherein one (26′) of the first and second coupling members is made of a magnetisable material.

8. The tool according to claim 1, including a detection and indication device (45) for detecting and indicating whether the key member (26″) has been drawn into the recess.

9. A drug administration set comprising the tool according to claim 1 and a drug supporting anchor (12) as the object to be manipulated,

10. The drug administration set according to claim 9, wherein the drug supporting anchor (12) is an intravaginal device and has a helical body, the flexible filament (28) being connected to one end of the helical body.

11. The drug administration set according to claim 10, further comprising a tube (10) adapted to accommodate said helical body in an extended configuration with reduced curvature, wherein said rod member (14) is adapted to be inserted into the tube (10) in order to push out the drug supporting anchor (12), and said first coupling member (26) is adapted to be pushed through the tube (10).

12. The drug administration set according to claim 11, wherein the rod member (14) has a handle (24) forming the second coupling member.

13. A method of drug administration, wherein a drug supporting anchor (12) is temporarily inserted into a body cavity, characterised by using a tool according to claim 1 for removing the drug supporting anchor from the cavity (16).

14. A method of drug administration, wherein a drug supporting anchor (12) is temporarily inserted into a body cavity, characterised by using a tool for removing the drug supporting anchor from the cavity (16);
wherein said tool is for manipulating a solid object (12) in a cavity (16) of a human or animal body, and comprises a longitudinal rod member (14) adapted for insertion into said body cavity (16), a first coupling member (26; 26′; 26″) connected to one of the rod member (14) and the object (12) through a flexible filament (28), and a second coupling member (24; 24′; 24″) connected to the other of the rod member and the object, said first and second coupling members being arranged for being mechanically coupled together through magnetic attraction, wherein one of the first and second coupling members forms a recess (30; 30′), the other of the first and second coupling members is configured as a key member (26; 26′; 26″), and magnetic poles (N, S) of at least one of the first and second coupling members are arranged for drawing the key member into the recess along a direction perpendicular to the longitudinal direction of the rod member, characterised in that the key member and recess are constituted such that when the key member is drawn into the recess, an operative contact surface (61) exists between the key member and the recess, the said contact surface extending in a direction perpendicular to the longitudinal direction of the rod member; and
wherein the drug administration set according to claim 11 is used for inserting the drug supporting anchor (12) into the body cavity (16) by inserting the tube (10) accommodating the drug supporting anchor (12) and then pushing the drug supporting anchor out of the tube (12) by means of the rod member (14) which is inserted into the tube (10) with its end opposite to the end forming the second coupling member (24).

15. The tool according to claim 1, wherein the second coupling member (24; 24′; 24″) forms the recess (30; 30′).

16. The tool according to claim 15, wherein the second coupling member (24; 24′; 24″) is rigidly connected to the rod member (14), and the recess (30; 30′) opens laterally of the rod member (14).

17. The tool according to claim 1, wherein the second coupling member (24; 24′; 24″) is rigidly connected to the rod member (14), and the recess (30;
30′) opens laterally of the rod member (14).

18. The tool according to claim 1, wherein each of the first and second coupling members (24, 26) includes a permanent magnet.

19. The tool according to claim 1, wherein at least one of the first and second coupling members includes an electro-magnet (42)

20. A method of drug administration, wherein a drug supporting anchor (12) is temporarily inserted into a body cavity, characterised by using a tool according to claim 8 for removing the drug supporting anchor from the cavity (16).
